(19) United States
(12) Patent Application Publication
(10) Pub. No.: US 2011/0105177 A1
(43) Pub. Date: May 5, 2011

(54) Cell/blood-monitor combination cellphone/blood glucose monitor

(76) Inventor: Rene M. Batiz, Beaumont, CA (US)

(21) Appl. No.: 12/803,936

(22) Filed: Dec. 9, 2010

Related U.S. Application Data
(60) Provisional application No. 61/271,094, filed on Jul. 17, 2009.

(51) Int. Cl. A61B 5/157; H04M 1/00

(52) U.S. Cl. 455/556.1; 600/365

(57) ABSTRACT

The external body of the invention is six and ¾ in length when flipped open and 3 inches when flipped close and 1¾ wide, all components parts including the external and internal will snap into place cut and design from harden plastic.

### BRIEF DESCRIPTION OF THE DRAWINGS

FIG. 1 Shows the invention Flipped open and its various components are illustrated and presented in relationship to one another.

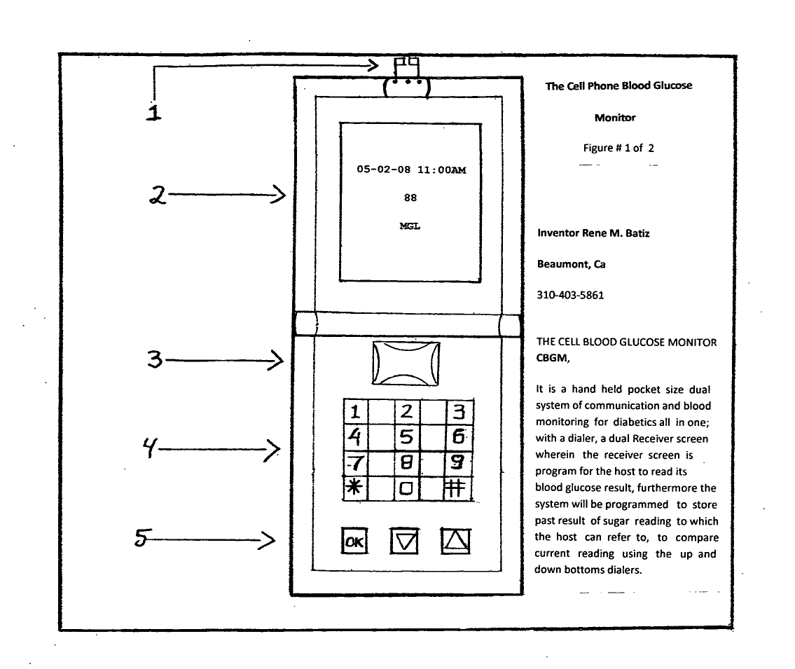

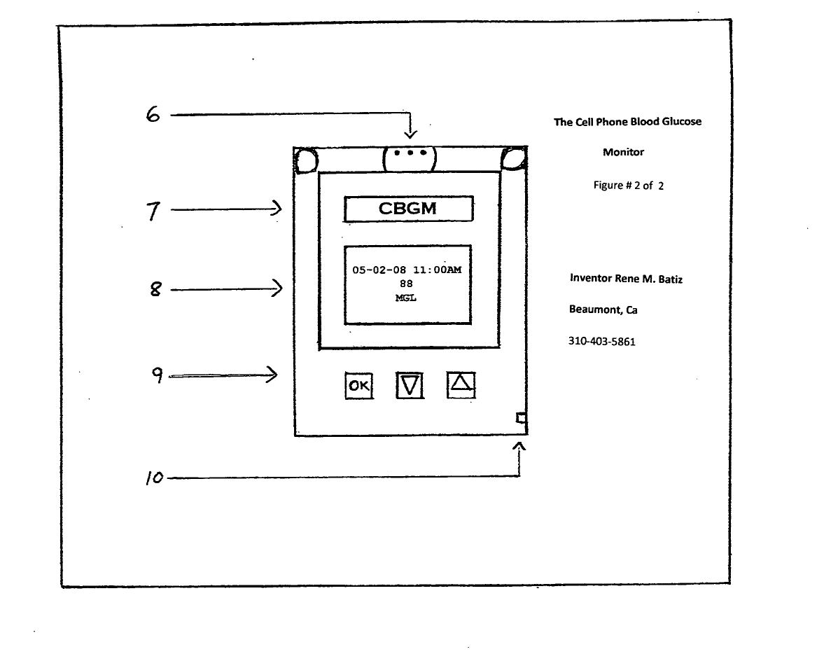

On FIG. 2 shows the invention flipped close displays additional component clearly illustrating the relationship to one another.

### DETAILED DESCRIPTION OF THE OF THE FIRST DRAWING

The Cell phone Blood Glucose Monitor it is a hand held pocket size dual system of communication and blood glucose monitoring for Diabetics subjects, combining the benefit of all that a cell phone can provide and the convenience of monitoring one sugar diabetes all in one product as illustrate in the following items ON FIGURE ONE. The invention is flipped open and in FIG. 2 it is flipped closed and clearly shows the name of invention in short (CBGM).

The invention is flipped open and it shows on top center location an open and tactile port is visible through where drops of blood is apply to read the level of sugar in the blood.

On this item the dual dialog screen box is clearly illustrated with date time and hour and the result of an current glucose reading; the dual display serves additional purpose as visual to reading names and phone number for incoming and outgoing calls.

This item portion of the invention which it is located just billow the hinges of the invention shows a four corner tactile tracker to find names and phone numbers in file memory of the Cell Phone Blood Glucose Monitor.

This item shows the digits Number from 1 to 0 (10) as well as the pound (*) symbol and the (#) number symbol all standards on cell phone for calling.

On this item are visible (3) bottoms on an square box just below the numbers; one displays an (OK) symbol which in this case its purpose is to stop and accept what one is searching for in the memory of Cell Phone Glucose monitor the other two (2) bottoms, Up and Down are the dialers to search for previous result of glucose results to be compare to the current one is testing for.

### DETAIL DESCRIPTION OF THE SECOND DRAWING

Here the invention it is clearly illustrate a flipped close Cell Phone Blood Glucose Monitor (CBGM) for short; which makes the size of the item and its proportion much more smaller than when it is open , here too the number of components and their purpose are clarified as follow.

The strip port is once again visible and tangible from where the drop of blood it is apply to read the result of the level of sugar in the blood.

Here the Name in short of the Cell Phone Blood Glucose Monitor CBGM is clearly displayed in bold and capital letters.

Here in this item the invention shows the dual display screen with date and time as well as the reading result of current sugar test in the blood by the host this result will be stored in the memory of the system for future comparison.

Here once again the (3) square bottoms with the OK symbol and the up and down dialers are clearly visible when the CBGM is flipped close giving the hosts a clear advantage to test and reads result without opening the CBGM.

This item is the electric charge port where from the battery will charge for daily use.

What it is claim:

1. The system claims when it is flipped open it reveals on the center top an open port for the placement of the strip tester where blood drops can be apply to begging the test of sugar in the blood. on FIG. 2 which shows the device flipped close from item 6 to 9, revels the name in short CBGM Cell phone Blood Glucose Monitor the dual dialog box and the up and down dialers; the number (10) item is electric port charger.

2. The system claims on the center top portion, an screen dialog dual display box is visible where the result of glucose test will be reveal in legible numbers, the screen also serves as visual reader for names and phones numbers of incoming and outgoing calls, and the time and date.

3. The system claims just below the hinge which serves to flip open or closing the device shows a four corner tactile to track names and phone numbers; just below that on figure (4) are number for dialing phone numbers follow by FIGURE (5) Which shows the up and down dialers that will serve to read the previous results of glucose reading saved in the memory of the system, the “Ok” bottom will serve as the stop dialer once comparison to current and previous reading is found.
